(19) United States
(12) Patent Application Publication
(10) Pub. No.: US 2022/0387811 A1
(43) Pub. Date: Dec. 8, 2022

(54) MEDICAL DEVICE AND METHOD FOR GENERATING A PLASMA-ACTIVATED LIQUID

(71) Applicant: Eberhard Karls Universität Tübingen Medizinische Fakultät, Tübingen (DE)

(72) Inventor: Martin Weiss, Mühlheim (DE)

(21) Appl. No.: 17/890,222

(22) Filed: Aug. 17, 2022

Related U.S. Application Data
(63) Continuation of application No. PCT/EP2021/053911, filed on Feb. 17, 2021.

(30) Foreign Application Priority Data
Feb. 18, 2020 (DE) 10 2020 104 261.2

(51) Int. Cl. A61N 1/44; H05H 1/24

(52) U.S. Cl. CPC A61N 1/44; H05H 1/2418; A61N 1/0524

(57) ABSTRACT

The present invention relates to a medical device for generating a plasma-activated liquid, a system for generating plasma-activated liquids comprising said device, and a method for generating a plasma-activated liquid. It also relates to a method for prophylaxis and treatment of postoperative adhesions.

### CROSS-REFERENCES TO RELATED APPLICATIONS

This application is a continuation of copending International Patent Application PCT/EP2021/053911 filed on 17 Feb. 2021 and designating the United States of America, which was not published in English, and claims priority of German Patent Application DE 10 2020 104 261.2 filed on 18 Feb. 2020, all of which are incorporated herein by reference.

### FIELD OF THE INVENTION

The present invention relates to a medical device for generating a plasma-activated liquid, a plasma-activated liquid generation system comprising said device, and a method for generating a plasma-activated liquid. It also relates to a method for prophylaxis and treatment of postoperative adhesions.

### BACKGROUND OF THE INVENTION

Physical plasma refers to a mixture of particles at the atomic-molecular level. Plasma was first described in the 1920s by chemist Irving Langmuir. It consists of a particle mixture of partially charged components, ions and electrons. Plasma can be generated artificially, for example by heating a neutral gas or exposing it to a strong electro-magnetic field until an ionized gaseous substance becomes increasingly electrically conductive.

Non-thermal or low temperature plasma can be generated at atmospheric pressure by an electrode system using a so-called dielectric barrier discharge. This is an AC gas discharge in which at least one of the electrodes is electrically isolated from the gas space by galvanic separation using a dielectric. A gas- or air-filled space between the insulatingly encased electrodes can then be ionized, or enters a plasma state, when an AC voltage across the electrodes generates sufficient field strengths in the gas space. The AC voltage required to generate a plasma is several kilovolts, i.e., a high voltage is required to generate a plasma. Pulsed excitation is also advantageous for the generation of a plasma, whereby voltage pulses with amplitudes in the kilovolt range with pulse durations of a few microseconds down to a few 10 nanoseconds are applied to the electrode arrangement.

A number of studies have demonstrated remarkable selective effects of low-temperature plasma on biological systems. For example, it is described that low-temperature plasma exerts an efficient antiseptic effect as well as a positive influence on chronic and acute wounds. It is also described that treatment of tumors with low-temperature plasma can lead to inactivation of cancer cells by initiation of apoptosis.

The so-called “biological reactive plasma factors” are responsible for the observed effects of plasma on biological systems. “Biologically reactive plasma factors” are held responsible for the observed effects of plasma on biological systems. These are essentially reactive species, such as reactive nitrogen species (RNS), reactive oxygen species (ROS), free radicals, ionic compounds such as NO₂⁻, NO₃⁻, ONOO⁻, electromagnetic radiation, etc., and possibly other species that have not yet been characterized in detail.

Increasingly, so-called plasma-activated liquids (PAL) or plasma-activated media (PAM) are being used. The antiproliferative, antineoplastic, and anti-inflammatory effects of plasma-activated liquids have recently been demonstrated by various studies. Plasma-activated liquids also have an efficient antiseptic effect as well as a positive influence on chronic and acute wounds. Plasma-activated liquids can significantly improve wound healing and reduce pain sensitivity. In contrast to the direct use of physical plasma, the use of plasma-activated liquids allows better dosage and control of the biologically reactive plasma factors to be applied.

### RELATED PRIOR ART

Devices for generating plasma-activated liquids are described, for example, in the following publications: Adachi et al. (2015), Plasma-activated medium induces A549 cell injury via spiral apoptotic cascade involving the mitochondrial-nuclear network, Free Radical Biology and Medicine 79, pp. 28-44; Kajiyama et al. (2017) Future perspective of strategic non-thermal plasma therapy for cancer treatment, J. Clin. Bio-chem. Nutr. 60, pp. 33-38; Nakamura et al. (2017) Novel intraperitoneal treatment with non-thermal plasma-activated medium inhibits metastatic potential of ovarian cancer cells, Nature Scientific Reports 7:6085, pp. 1-14; and Azzariti et al. (2019), Plasma-activated medium triggers cell death and the presentation of immune activating danger signals in melanoma and pancreatic cancer cells, Nature Scientific Reports 9:4099, pp. 1-13.

The known devices are used to activate liquids by treating them with plasma. The known devices use external plasma generators. They are therefore difficult to handle and not very suitable for direct clinical use by an operator. For this reason, the devices used to date for generating plasma-activated liquids are used exclusively in experimental systems.

Current devices also do not allow for immediate application of the plasma-activated liquid to the tissue. Rather, it is produced at an initial time and used on the tissue at a later time. In the prior art, plasma-activated liquids are prepared “in stock,” so to speak. However, the biologically reactive plasma factors are “volatile”, i.e., stable only for a limited time. The plasma-activated liquids currently produced are therefore often of little therapeutic value, since storage often leaves only low concentrations of biologically reactive plasma factors.

US 2008/0292497 discloses a large scale device for disinfecting a liquid via plasma impingement. A first gas-containing compartment is separated from a second compartment via a phase separator. The phase separator may be a porous or non-porous membrane with holes to allow gas transfer from the first compartment to the second compartment. The gas is pumped into the second compartment by applying a positive pressure to the first compartment. This causes gas bubbles to form in the liquid in the second compartment in the form of macrobubbles. This device is not only unsuitable for a medical application because of its large scale. Because of the accumulation of gas bubbles, there is a risk that they could be washed into the blood system and cause embolisms. Use in endoscopic procedures such as hysteroscopy is also out of the question, as the gas bubbles formed make visibility difficult.

DE 10 2014 105 720 discloses an active layer for inactivating bacteria on surfaces or for biomedical application. In this process, plasma-generated species are transferred from a discharge space into an active medium located in a space adjacent thereto. The discharge space and the space containing the active medium are separated by an uncharacterized membrane. This device is also unsuitable for the prophylaxis and treatment of postoperative adhesions.

### SUMMARY OF THE INVENTION

Against this background, the problem underlying the invention is to provide a medical device for generating a plasma-activated liquid, with which the disadvantages of the current devices from the prior art can be avoided or at least reduced. In particular, it is intended to provide such a medical device which can be easily handled by a treating physician and which can be manufactured on an industrial scale. It should also preferably allow the plasma-activated liquid to be used immediately after its production, without the need for prolonged storage.

This problem is solved by a medical device for generating a plasma-activated liquid, comprising a plasma discharge space and a liquid-carrying space adjacent thereto to form an interface, the interface comprising a semipermeable membrane permeable to biologically reactive plasma factors from the plasma discharge space and impermeable to the liquid from the liquid-carrying space.

By separating the plasma discharge space from the liquid-carrying space by means of a semipermeable membrane, the design requirements for a medical device that is easy to handle for the treating physician and can be manufactured on a large scale are created in an advantageous manner.

The plasma discharge space is configured to receive a medium suitable for generating a physical plasma, such as a gas, for example argon, helium, O₂, N₂, room air or other suitable gases.

The liquid-carrying space is configured to receive a liquid, such as water, saline solution, biological buffers, etc.

The semi-permeable membrane, which can be realized, for example, by a conventional dialysis membrane, allows defined, mutually delimited plasma discharge and liquid-carrying spaces to be created. At the same time, it is ensured that the biologically reactive plasma factors accumulate in the liquid and can migrate from the plasma through the semipermeable membrane into the liquid.

In one embodiment of the invention, the semipermeable membrane is configured in such a way that the formation of gas bubbles, preferably macro-bubbles, further preferably macroscopically visible macro-bubbles, i.e. those with a diameter in the centimeter or millimeter range, is prevented in the liquid-carrying space. According to this embodiment, the skilled person will adjust the pore diameter by suitable selection of the material of the semipermeable membrane in such a way that essentially only the biologically reactive plasma factors are transferred from the plasma discharge space into the liquid-carrying space. This is the case with conventional dialysis membranes or conventional biologically symmetrical dialysis membranes, such as cuprophan, hemophane, or cellulose triacetate, as well as other membranes based on the natural polymer cotton cellulose. The average pore radius of conventional dialysis membranes is 1.72 nm, which corresponds to a permeability barrier of 1000, preferably 500 Daltons, i.e., larger molecules cannot penetrate the membrane; cf. Nowack et al. (2019), Dialysis and Nephrology for Professionals, 3rd edition, chapter 7, Structure of Dialyzers, pp. 89-103. The use of conventional dialysis membranes according to the invention thus prevents (gas) bubbles from forming in the liquid of the liquid-carrying compartment.

In another embodiment of the invention, the semipermeable membrane has an average pore radius of <5 nm, preferably of 2 nm. This ensures that not only the formation of macrobubbles in the liquid but even microbubbles is physically impossible.

According to the invention, the plasma discharge space can be realized by a continuous cavity, in particular in the case of a tubular or hose-shaped configuration of the device according to the invention, in which the plasma discharge space forms an outer tubular or hose body and the liquid-carrying space forms an inner tubular or hose body forms or alternatively in the reverse arrangement, spacers can be provided which ensure a spacing of the plasma discharge space from the liquid-carrying space. Alternatively, the plasma discharge space can be created by or include a gas-permeable material, such as glass fiber fabric or plastic. This has the advantage that the arrangement of the plasma discharge and liquid-carrying spaces can be implemented in a flexible manner in a layered construction and the provision of spacers is unnecessary.

The object on which the invention is based is hereby completely achieved.

The inventor provides for the first time a device for the generation of plasma-activated liquids that is easy to handle and produce, with which inflammatory, chronic-inflammatory, neoplastic and oncological diseases can be treated intracorporeally in an advantageous manner. The plasma-activated liquid can be used immediately after it has been generated on the tissue, which ensures a high degree of effectiveness.

The device according to the invention is also ideally suited for the prophylaxis and treatment of postoperative, iatrogenic adhesions or those caused by inflammatory diseases. These pose major challenges to the healthcare system during surgical or endoscopic/laparoscopic procedures. Due to the antiproliferative, anti-inflammatory, and wound-healing properties of plasma-activated liquids, the use of the device according to the invention is suitable as a routine therapeutic procedure during the completion of surgery to prevent postoperative adhesions.

Its non-invasiveness and non-toxicity predestines the device according to the invention for intracorporeal application. Compared to the local application of physical plasma, the medical device according to the invention enables large-area and uniform treatment of body cavities, such as the abdominal or vaginal cavity, the oral and pharyngeal cavities, the thorax, gastrointestinal cavity, joint spaces, etc.

In one embodiment, the pressure conditions in the plasma discharge gap can be selectively controlled by providing or connecting a suitable device. For example, a low pressure or even vacuum or “near-vacuum” can prevail in the plasma discharge gap. This measure has the advantage that the energy and voltage required for plasma generation would be significantly reduced and a more homogeneous plasma could be generated.

In one embodiment of the medical device according to the invention, a positive electrode insulated with a dielectric is adjacent to the plasma discharge space on a side opposite the interface.

This measure creates the constructive conditions for the generation of plasma by dielectric barrier discharge.

The structure of the positive electrode may be ring-shaped, grid-shaped, spindle-shaped, meander-shaped, or honeycomb-shaped. These embodiments create a uniform distribution of the electrode structure, which ensures a very homogeneous plasma generation in the plasma discharge space. They also allow for advantageous flexibility or shape adaptability of the electrode structure.

The dielectric may be formed of glass, ceramic or plastic, for example silicone. In particular, if the dielectric is formed of a soft plastic such as silicone, it may have flexibility, i.e., pliability, which is advantageous in the application.

In an alternative embodiment, the electrode structure is not insulated with a dielectric towards the plasma discharge space, thereby enabling a discharge, for example, in the form similar to a corona discharge.

In a further embodiment of the medical device according to the invention, a ground electrode is arranged in the plasma discharge space on and/or in the vicinity of the interface. In this regard, the ground electrode may be integrated into the interface.

In this embodiment, the ground electrode constitutes the counter-electrode to the positive electrode, thereby creating the structural conditions for the generation of plasma by dielectric barrier discharge.

The structure of the ground electrode may correspond to that of the positive electrode.

In this embodiment, the biologically reactive plasma factors can migrate from the plasma through the membrane into the liquid by passive diffusion due to Brownian motion. This ensures membrane-sparing migration of the biologically reactive plasma factors. Thereby, the enrichment of the liquid with the reactive plasma factors can be regulated by the volume per time of the liquid flowing through or the energy input of the plasma and thus be adapted to the clinical conditions.

The spacing of the positive electrode from the ground electrode is selected in such a way that, on the one hand, heat generation is avoided, and, on the other hand, sufficient physical plasma can form between the positive electrode and the ground electrode. This can be achieved by the above-mentioned spacers and/or by designing the plasma discharge space from a gas-permeable material with sufficient thickness.

In another embodiment of the device according to the invention, a ground electrode is arranged within the liquid-carrying space.

According to the invention, the arrangement of the ground electrode “within” the liquid-carrying space means that the ground electrode is arranged in such a way that it can come into contact with the liquid at least on one side, preferably on both sides or completely. In this embodiment, the biologically reactive plasma factors can migrate into the liquid in an accelerated manner not only due to Brownian motion but rather charge-driven from the plasma through the membrane. This arrangement thus ensures rapid and strong enrichment of the liquid with biologically reactive plasma factors. The enrichment of the liquid with the reactive plasma factors can be regulated and adapted to the clinical conditions not only by the volume per time of the liquid flowing through but also by the applied voltage.

Also in this embodiment, the spacing of the positive electrode from the ground electrode is thereby selected such that, on the one hand, heat generation is avoided and, on the other hand, sufficient physical plasma can form between the positive electrode and the ground electrode. This can be achieved, as mentioned, by using the spacers discussed above and/or by designing the plasma discharge space from a gas-permeable material of sufficient thickness.

In another embodiment, the medical device according to the invention is tubular and/or hose-shaped.

This measure has the advantage that the tubular and/or hose-like shape makes it possible in a simple manner to delimit the individual spaces and structures from one another and to electrically insulate the electrode structures. In addition, a tubular and/or hose shape of the device according to the invention simplifies the flexible and bendable design. Also, a tubular and/or hose-like shape of the device according to the invention simplifies access into body cavities through minimally invasive approaches, as well as application into body cavities and/or openings, which also often have a tubular and/or hose-like shape. According to the invention, “tubular and/or hose-shaped” means that the various spaces and structures are arranged in the form of nested tubes or hoses. This embodiment form can be integrated particularly well into existing modules, such as endoscopic devices, high-pressure nebulization and/or spraying units, etc.

Thus, in a first tubular and/or hose-shaped embodiment, the liquid-carrying space is located in the innermost part of the device, which is bounded on the outside by the semi-permeable membrane, on which in turn the ground electrode is arranged on the outside, which is bounded on the outside by the plasma discharge space via spacers. This is bounded on the outside by the dielectric and the adjoining positive electrode. The positive electrode can in turn have an outer insulation on its outer side. The very outer-most layer may be formed by a support material, which provides structure and possibly flexibility to the device.

In another tubular and/or hose-shaped embodiment, the ground electrode is located in the innermost part of the device and is surrounded by the liquid-carrying space. The liquid-carrying space is bounded on the outside by the semi-permeable membrane, which in turn is bounded on its outside by the plasma discharge space. This is bounded on its outside by the dielectric and the adjoining positive electrode. The positive electrode can in turn have an outer insulation on its outer side. The very outermost layer may be formed by a support material, which provides structure and possibly flexibility to the device.

In a still further embodiment, the positive electrode is located in the innermost part of the device according to the invention. Around this, the plasma discharge space is bordered on the outside, and on the outside of this space, in turn, the liquid-carrying space is provided. The further construction of this still further embodiment corresponds to that of the aforementioned embodiments.

In a further embodiment, the device according to the invention is box-shaped.

This alternative embodiment can advantageously make use of the sandwich construction method, whereby the - now horizontal - arrangement and sequence of the spaces and structures essentially correspond to those described for the tubular and/or hose-shaped embodiments. This embodiment also integrates well with existing modules.

In another embodiment of the invention, the medical device comprises a support structure enclosing it.

This measure has the advantage of providing a structure that gives the device structure and, if desired, flexibility or even rigidity. Further, the carrier provides an additional barrier to ensure that the generated plasma can be applied in a targeted manner to the tissue to be treated and that other areas of the body cannot come into contact with the plasma. The carrier can be made of metal or plastic, for example.

In one embodiment, the medical device according to the invention has a gas connection via which a carrier gas can be introduced into the plasma discharge space and, if necessary, released again.

By this measure, a gas, for example argon gas, can be introduced into the plasma discharge space, which is well suited for the generation of a plasma, for example by dielectric barrier discharge.

In one embodiment, the medical device according to the invention comprises a connection for a high pressure nebulization and/or spraying unit.

By this measure, the device according to the invention is integrated into an apparatus that allows a targeted and, if necessary, areal application of the plasma-activated liquid. Thus, the connection of the device according to the invention to a high-pressure nebulization and/or spraying unit enables the targeted treatment of tumor tissue. Postoperative adhesions can also be reduced or avoided, for example, by fogging and/or spraying surgically treated tissue with the plasma-activated liquid. This can be done during or directly after the surgical procedure, so that postoperative follow-up treatment can preferably be avoided.

In another embodiment of the invention, the medical device has a connector for connection to an endoscopic device.

This measure has the advantage that the device according to the invention can be integrated into endoscopic operations and into existing endoscopic systems or trocars, allowing their use immediately during or following the surgical procedure.

According to one embodiment, the medical device according to the invention is configured for intermittent and/or continuous generation of a plasma activating liquid.

This embodiment can be realized, for example, by connecting a pump or comparable device for conveying the liquid, which conveys it optionally intermittently and/or continuously through the liquid-conducting space. Alternatively, the plasma discharge can also be controlled by intermittent and/or continuous voltage application. This measure allows the setting of a suitable treatment mode depending on the respective purpose of use.

Another object of the invention relates to a system for generating plasma-activated liquids with the medical device according to the invention and with a high-voltage source connectable to the medical device for applying high voltage to the electrode(s).

The features, embodiments and advantages of the device according to the invention apply accordingly to the system according to the invention.

Another object of the invention relates to a method for generating a plasma-activated liquid, comprising the following steps:
1. Providing a device comprising a plasma discharge space and a liquid-carrying space adjacent thereto to form an interface, the interface comprising a semi-permeable membrane permeable to biologically reactive plasma factors from the plasma discharge space and impermeable to the liquid from the liquid-carrying space;
2. Flowing a gas through the plasma discharge space,
3. Flowing a liquid through the liquid-carrying space,
4. Generating a physical plasma containing biologically reactive plasma factors from the gas in the plasma discharge space,
5. Allowing the biologically reactive plasma factors to migrate through the semi-permeable membrane into the liquid.

The features, embodiments and advantages of the device according to the invention apply accordingly to the process according to the invention.

The gas may preferably be argon gas, helium, O₂, N₂, room air or other suitable gases.

“Allowing the biologically reactive plasma factors to migrate” generally refers to the movement of the biologically reactive plasma factors through the semipermeable membrane. In particular, it includes passive diffusion of the biologically reactive plasma factors through the semipermeable membrane due to Brownian motion, as well as charge-driven motion diffusion of the biologically reactive plasma factors through the semipermeable membrane.

In one embodiment of the method according to the invention, the device provided is the medical device according to the invention.

Another object of the present invention relates to the use of a plasma-activated liquid in the prophylaxis and/or treatment of postoperative adhesions.

Preferably, the plasma-activated liquid is one that has been produced using the device according to the invention and/or the method according to the invention.

The features, embodiments and advantages of the device according to the invention and the process according to the invention apply accordingly to the use according to the invention.

Further advantages and features are apparent from the following description of preferred embodiments and the accompanying drawings.

It is understood that the above-mentioned features, which will be explained below, can be used not only in the combination indicated in each case, but also in other combinations or on their own, without departing from the scope of the present invention.

### BRIEF DESCRIPTION OF THE DRAWINGS

FIG. 1 shows a first (A) and second (B) embodiment of the device according to the invention in longitudinal section;

FIG. 2 shows a first embodiment of the device according to the invention (A) in broken-up plan view and (B) in cross-section;

FIG. 3 shows a second embodiment of the device according to the invention (A) in broken-up plan view and (B) in cross section;

FIG. 4 shows a third embodiment of the device according to the invention in broken-up plan view;

FIG. 5 schematically shows the use of the device according to the invention during a gynecological operation; and

FIG. 6 Selective PAL effects on mesothelial cells and fibroblasts. a) PAL dose-de-pendent proliferation of human fibroblasts and mesothelial cells. PAL doses of 1:2 result in specific inhibition of fibroblasts, with continued proliferation of mesothelial cells. b) Bright field microscopy of fibroblasts (upper panel) and mesothelial cells (lower panel). c) Hydroxyproline assay (left) and Sircol assay (right) for quantification of extracellular insoluble collagen and procollagen. PAL treatment results in a decreased amount of insoluble collagen with an increased amount of soluble procollagen. d) Flow cytometry after PI staining. A PAL dose of 1:2 results in fibroblast-specific G1 cell cycle arrest. e) Cell viability assay. A PAL dose of 1:2 results in a fibroblast-specific reduction in cellular metabolism inducing various cellular mechanisms and an increase in cellular metabolism in mesothelial cells.

### DESCRIPTION OF PREFERRED EMBODIMENTS

1. Details of the Device according to the Invention.

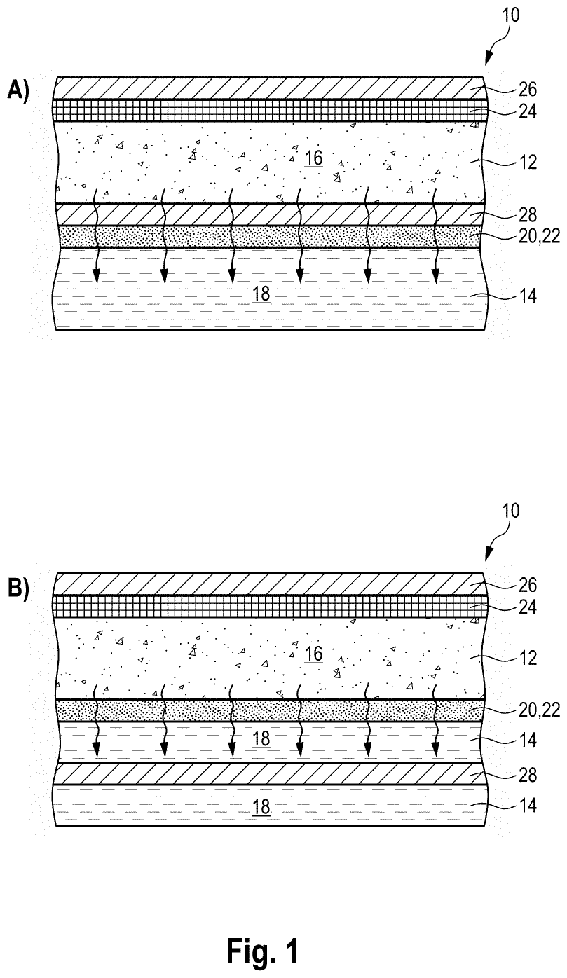

FIG. 1A shows an enlarged longitudinal section through a medical device for generating a plasma-activated liquid, designated by the general reference sign 10. The device 10 is suitable for intracorporeal treatment of inflammatory, chronic inflammatory, neoplastic, and oncological diseases, as well as for postoperative adhesion prophylaxis. The medical device 10 has a plasma discharge space 12 and a liquid-carrying space 14 adjacent thereto at the bottom in the embodiment. Physical plasma 16, particularly low or room temperature plasma under normal atmospheric pressure or even under low pressure conditions, can be generated in the plasma discharge space 12. A liquid 18, such as water, a buffer solution or a physiological saline solution, flows intermittently or continuously through the liquid-carrying space 14.

The plasma discharge space 12 and the liquid-carrying space 14 adjacent to the underside thereof form an interface 20 that includes a semipermeable membrane 22. The semipermeable membrane 22 is permeable to biologically reactive plasma factors from the plasma 16 formed in the plasma discharge space 12 and impermeable to the liquid 18 from the liquid-carrying space 14.

A positive electrode 26, insulated from the plasma discharge space 12 with a dielectric 24, is adjacent to the top surface of the plasma discharge space 12 as shown in the embodiment. A dissipative ground electrode 28 is adjacent to the semipermeable membrane 22 with orientation toward the plasma discharge space 12. The electrodes 26 and 28 may be formed by a single wire or may be lattice-, spindle-, meander-, or honeycomb-shaped.

When high voltage is applied to the electrodes 26 and 28, a physical plasma 16 is generated in the plasma discharge space 12. The biologically reactive plasma factors therein can migrate through the semipermeable membrane 22 into the liquid 18 in the liquid carrying space 14 due to Brownian motion, as indicated by the serpentine arrows.

FIG. 1B shows a second embodiment in which the structures and features corresponding to those of FIG. 1A are shown with the same reference signs. The embodiment shown in FIG. 1B differs from the embodiment shown in FIG. 1A in that now the ground electrode 28 no longer rests against the upper side of the semipermeable membrane 22 with orientation towards the plasma discharge space 12, but is arranged inside the liquid-carrying space 14. When high voltage is applied to the electrodes 26 and 28, the biologically reactive plasma factors now no longer migrate through the semipermeable membrane 22 into the liquid-carrying space 14 solely due to Brownian motion, but are “shot into” it in a charge-driven manner.

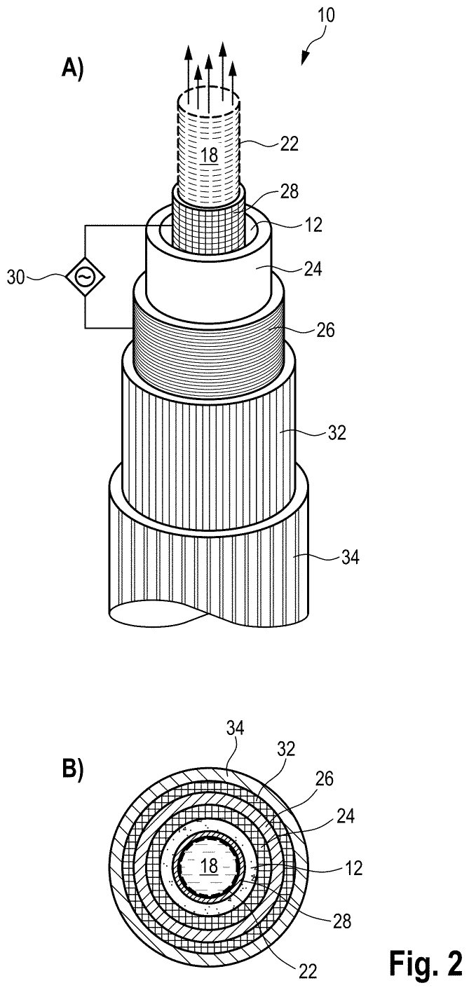

In FIG. 2, the medical device 10 according to the invention is shown according to a first embodiment (corresponding to the arrangement shown in FIG. 1A). Sub-figure A shows the device 10 in a broken-up plan view, and sub-figure B shows a cross-section through the device 10. Structures and features corresponding to those shown in FIGS. 1A and 1B are shown with the same reference signs. In addition, in this embodiment, the device 10 according to the invention has a high-voltage source 30 for applying high voltage to the electrodes 26 and 28, an outer insulation 32 insulating the positive electrode 26 from the outside, and a carrier 34 surrounding this outer insulation.

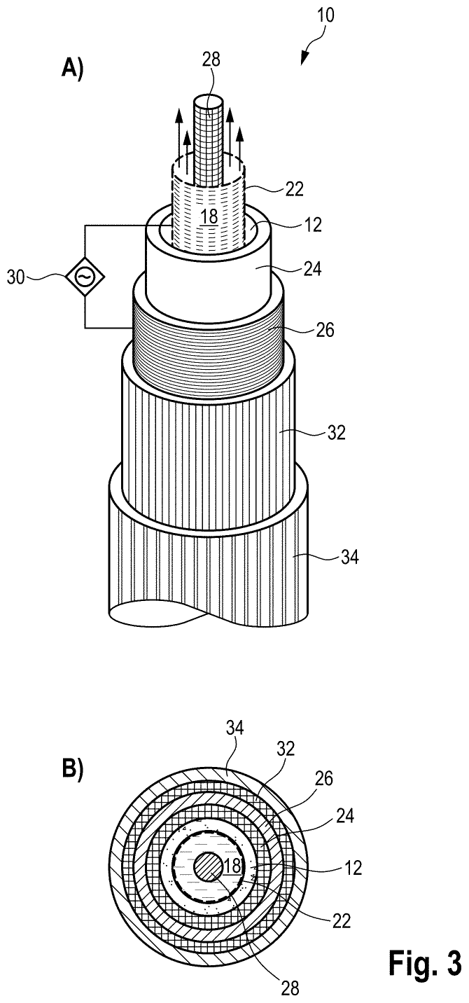

In FIG. 3, the medical device 10 according to the invention is shown according to the second embodiment (corresponding to the arrangement shown in FIG. 1B). Sub-figure A shows the device 10 in a broken-up plan view, sub-figure B shows a cross-section through the device 10. Structures and features corresponding to those of FIG. 2 are shown with the same reference signs.

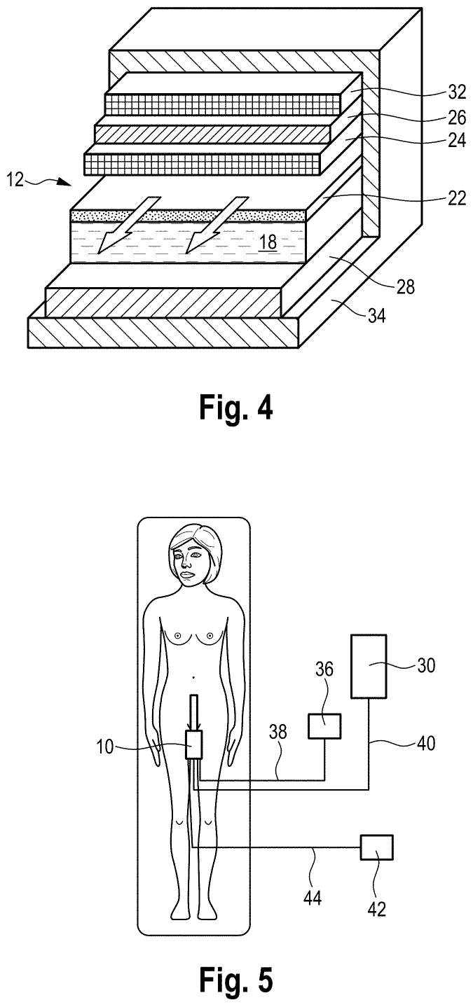

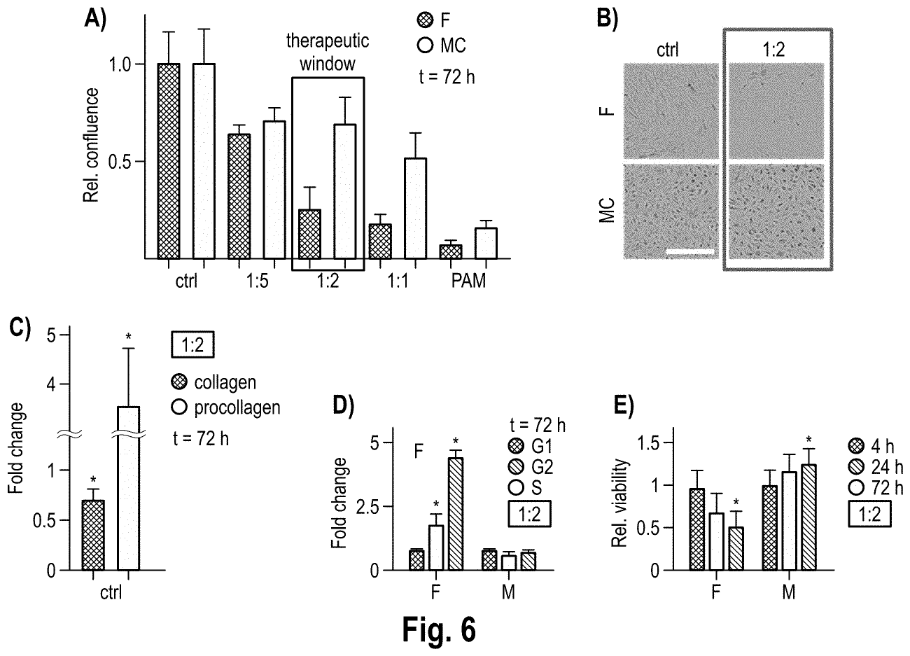

In FIG. 4, the medical device 10 according to the invention is shown in a third embodiment, in which the structures and spaces are arranged in a sandwich-like manner in horizontal layering in a carrier 34 with a box-like design. Structures and features corresponding to those of FIGS. 1, 2 and 3 are shown with the same reference signs.

FIG. 5 schematically shows the use of the device 10 according to the invention during a gynecological operation. Also shown are a pump 36 connected to the device according to the invention for supplying liquid via a hose 38, a high-voltage source or high-voltage generator 30 for applying high voltage to the electrodes via a cable 40, and a gas connection with a gas source 42 and a gas line 44, via which a carrier gas can be introduced into the plasma discharge space 12 and, if necessary, discharged again.

### 2. Plasma-Activated Liquid Enables Specific Inhibition of ECM-Producing Connective Tissue Cells for Prophylaxis of Postoperative Adhesions—Experiments

Gynecologic and general surgery below the transverse colon carries a particularly high risk for postoperative adhesions (PA) and associated severe disease. Clinically, PAs are often characterized by chronic severe pain syndromes in the abdomen, flanks, or back that are often misdiagnosed for years. PAs also account for 15-20% of all cases of secondary infertility and 50-70% of all mechanical ileus. PAs are estimated to cause enormous costs to health care systems. The cause of PA is excessive extracellular matrix (ECM) formation due to activation of peritoneal mesothelial cells, fibroblasts and immune cells. Plasma activated liquid (PAL) could prevent PA by inhibiting the dysregulation and overproliferation of fibrin and ECM-producing connective tissue cells.

Dose-dependent PAL treatment of primary human mesothelial cells and fibroblasts with the device of the invention showed a defined and reproducible therapeutic window (denoted here as 1:2) (FIG. 1a,b). With this PAL concentration, the excessive cellular proliferation of ECM- and fibrin-producing fibroblasts could be significantly inhibited, whereas the physiological cell proliferation of mesothelial cells did not change significantly. Extracellular amounts of soluble procollagen (less cross-linked) were significantly increased after PAL treatment, whereas insoluble (highly cross-linked) collagen significantly decreased. The selective antiproliferative effect on primary fibroblasts was associated with significant G2 cell cycle arrest and a significant decrease in cellular viability. Interestingly, the same PAL dose showed a significant increase in mesothelial cell viability.

Accordingly, peritoneal PAL treatment using the device of the invention offers a hopeful medical application to selectively reduce postoperative (over)proliferation of ECM and fibrin-producing fibroblasts, as well as synthesis and cross-linking of functional ECM components such as collagen.

Therefore, what is claimed, is:

1. A medical device for generating a plasma-activated liquid, comprising a plasma discharge space and a liquid-carrying space adjacent thereto to form an interface, characterized in that the interface comprises a semipermeable membrane permeable to biologically reactive plasma factors from the plasma discharge space and impermeable to the liquid from the liquid-carrying space.

2. The medical device according to claim 1, wherein the semipermeable membrane is configured in such a way that in the liquid-carrying space in the liquid the formation of gas bubbles is prevented.

3. The medical device according to claim 2, wherein the gas bubbles are macrobubbles.

4. The medical device according to claim 1, wherein the semipermeable membrane has an average pore radius of about <5 nm.

5. The medical device of claim 4, wherein the semipermeable membrane has an average pore radius of about ≤2 nm.

6. The medical device according to claim 1, wherein the semipermeable membrane has an exclusion limit of about 1000 Daltons.

7. The medical device according to claim 1, wherein the semipermeable membrane has an exclusion limit of about 500 Daltons.

8. The medical device according to claim 1, wherein a positive electrode insulated with a dielectric is adjacent to the plasma discharge space on a side opposite the interface.

9. The medical device according to claim 1, wherein a ground electrode is arranged in the plasma discharge space on and/or near the interface.

10. The medical device according to claim 1, wherein a ground electrode is disposed within the liquid carrying space.

11. The medical device according to claim 1, which is tubular and/or hose-shaped.

12. The medical device according to claim 1, which is box-shaped.

13. The medical device according to claim 1, which comprises an enclosing support structure.

14. The medical device according to claim 1, which comprises a gas connection via which a carrier gas can be introduced into plasma discharge space.

15. The medical device according to claim 14, which comprises the gas connection via which the carrier gas can be released from the plasma discharge space.

16. The medical device according to claim 1, which comprises a connection for a high-pressure nebulization or spraying unit.

17. The medical device according to claim 1, which comprises a connector for connection to an endoscopic device.

18. The medical device according to claim 1, which is configured for intermittent or continuous generation of a plasma-activating liquid.

19. A system for generating plasma activated liquids, comprising the medical device according to claim 8, and a high voltage source connectable to the medical device for applying high voltage to the electrode.

20. A method for generating a plasma activated liquid comprising the steps of:
1. Providing a device having a plasma discharge space and a liquid-carrying space adjacent thereto to form an interface, the interface comprising a semipermeable membrane permeable to biologically reactive plasma factors from the plasma discharge space and impermeable to the liquid from the liquid-carrying space;
2. Flowing a gas through the plasma discharge space,
3. Flowing a liquid through the liquid-carrying space,
4. Generating a physical plasma containing biologically reactive plasma factors from the gas in the plasma discharge space,
5. Allowing the biologically reactive plasma factors to migrate through the semi-permeable membrane into the liquid.

21. The method according to claim 20, wherein the device provided is the device according to claim 1.

22. A plasma-activated liquid for use in the prophylaxis and/or treatment of postoperative adhesions.
